(19) United States
(12) Patent Application Publication
(10) Pub. No.: US 2008/0200773 A1
(43) Pub. Date: Aug. 21, 2008

(54) Catheter and Portable Data Managing Device

(76) Inventor: Gheorghe Aurel Marie Pop, Nijmegen (NL)

Correspondence Address: HARNESS, DICKEY & PIERCE, P.L.C., P.O. BOX 8910, RESTON, VA, 20195, US

(21) Appl. No.: 11/597,292

(22) PCT Filed: May 26, 2005
(86) PCT No.: PCT/EP2005/005819

§ 371 Date: Dec. 26, 2007

(30) Foreign Application Priority Data
May 26, 2004 (NL) 1026274

(51) Int. Cl. A61B 5/0215

(52) U.S. Cl. 600/301

(57) ABSTRACT

An assembly of a catheter and a portable data managing device is disclosed. The catheter includes a tubular body with a distal end portion to be introduced into the body of a person and a proximal end portion, including at its distal end portion at least one sensing member and at least one connecting device extending to the proximal end portion for connecting the sensing member to the data managing device. The data managing device includes a control and processing device for controlling the at least one sensing member and processing signals generated thereby.

The invention relates to an assembly of a catheter, comprising sensing means and a device for controlling the sensing means and processing signals generated by the sensing means.

Assemblies of this kind are known and are for instance used for determining the pressure inside a blood volume in a patient=s body, or for measuring the impedance of the blood of the patient in a ventricle of the heart. See e.g. PCT/NL00/00378.

The device according to the invention as characterized in claim 1 has the specific advantage that a patient, using this assembly, is not confined to a hospital bed, but can walk around and more or less follow his or her daily routines.

In this way, it is possible to monitor the patient for a considerable longer period of time than would be normally possible when the patient were to be hospitalized.

The portable data managing device of the assembly according to the invention, can be made small by using the features of claim 2. The portable data managing device in that case only contains the most elementary parts and a large part of the data processing can be done by the computer device.

In a further development the assembly according to the invention is provided with interfacing means for interfacing the processing means with a telecommunication device. Relevant data can, in this way, be transmitted to for instance a doctor, who is treating the patient.

The assembly according to the invention can be used for a great number of monitoring functions. For example, the assembly can be designed or comprise means for measuring impedance in a body fluid, for example in a volume of blood. Another possibility is to measure temperatures inside the body of the patient.

Furthermore, the measuring of a pressure in a body fluid and the measuring of the concentration of at least one chemical component in a body fluid is possible with the device according to the invention.

If the catheter is of the kind to be introduced into the heart of a patient, the sensing means can be designed for measuring the electrical activity of the heart, and can be used for registering a ECG. This feature can also very advantageously be used for positioning the distal end of the catheter in the proper position in the heart by checking the ECG while introducing the catheter.

The invention will be further illustrated in the following description of a preferred embodiment, with a reference to the drawings.

In the drawings,

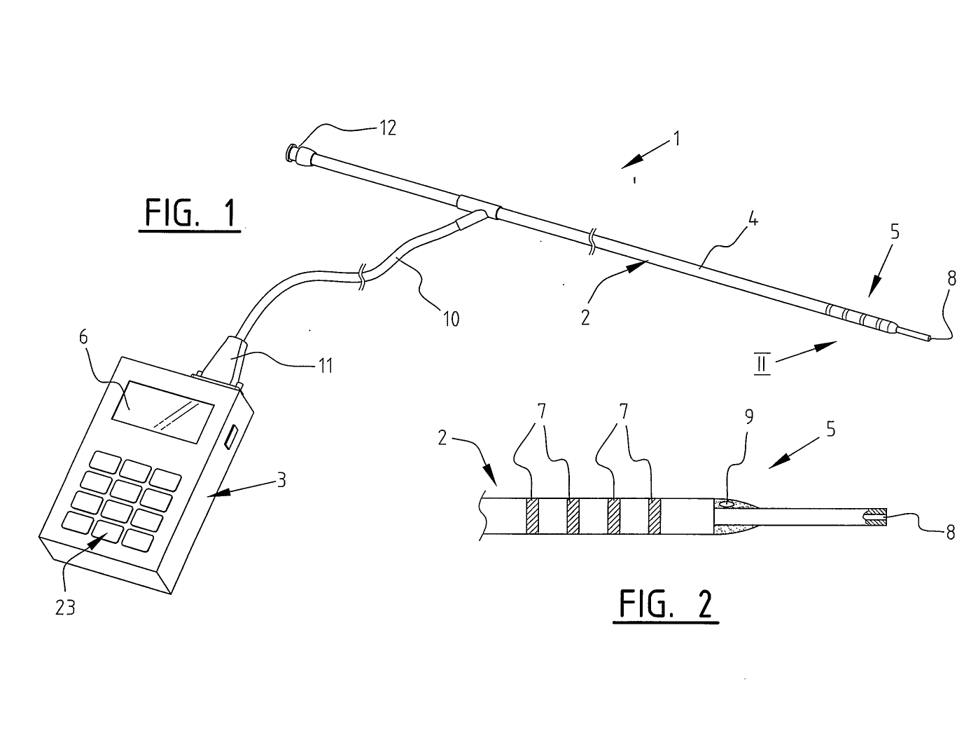

FIG. 1 shows a simplified perspective view of the assembly according to the preferred embodiment of the present invention.

FIG. 2 shows a detail of FIG. 1, according to II in FIG. 1.

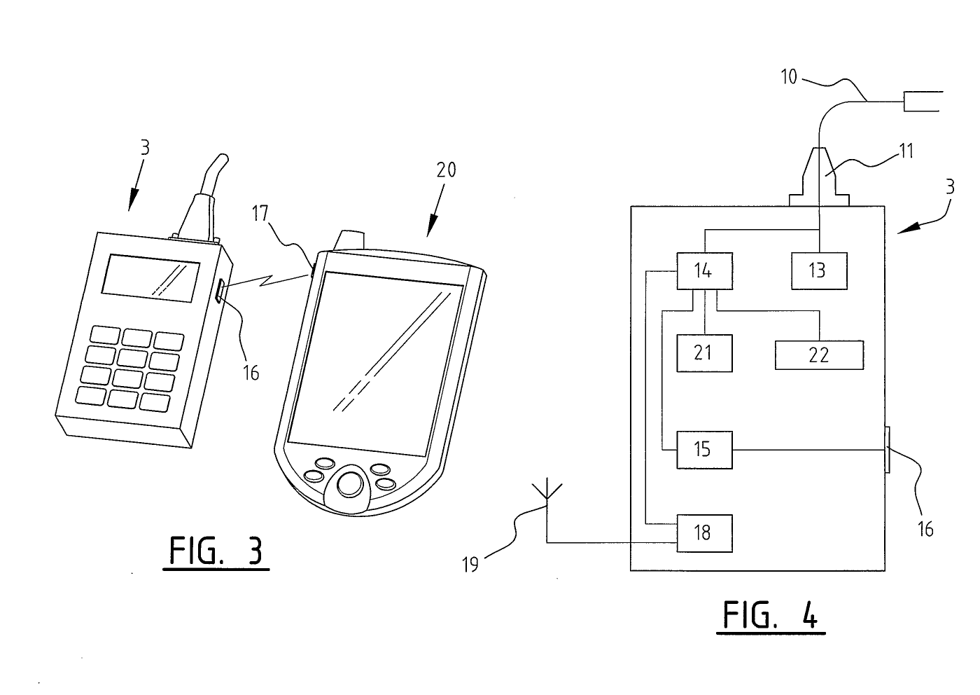

FIG. 3 illustrates the use of the data management device of the assembly according to the invention, together with a portable computer device.

FIG. 4 shows a schematic view of the components of the data managing device according to the invention.

The assembly shown in FIG. 1 comprises a catheter 2 and a data managing device 3.

The catheter 2 is provided with a distal portion 5 intended to be introduced in the body of a patient. At the distal portion 5, the catheter 2 comprises in this embodiment four electrodes 7 and a thermistor 9 as sensing means. Each of these sensing members is connected by connecting means which run through the proximal end of the catheter 2, through the lead 10 to the connector 11, connecting these sensing means with the data managing device 3.

The catheter in this embodiment is provided with one lumen 8 extending from the distal end to the proximal end. At the proximal end a connecting device such as a luer lock is provided for connecting for example a fluid supply to the lumen 8. In this way, fluids such as medicines can be supplied to the body of the patient at the position of the distal end of the catheter. The data managing device can be used also to control and to manage the fluid and medicine supply.

The data managing device 3 is in the embodiment shown, provided with a display screen 6 and a key pad 23. On the display screen 6, instructions and/or measurement data can be shown.

The key pad can be used for starting certain functions of the device or, for instance, selecting data to be shown in the display screen 6.

As schematically shown in FIG. 4, the data managing device of the assembly according to the invention comprises control means 13, for controlling the sensors at the distal end of the catheter. If, for example, the electrodes 7 are used for measuring the impedance in a cavity of the heart of the patient, the control means 13 will be programmed such that an electrical alternating current is generated between two of the electrodes and electrical signals generated thereby in the other two electrodes will be collected and further processed. In this regard reference is made to PCT/NL00/00378, describing in detail the measuring of impedance with a catheter of this kind.

The data managing device 3 further comprises processing means 14, processing the measurement signals generated by a sensing means.

With the processing means 14, data storage means 21 are connected, for storing measurement data for a certain period of time.

The device 3 is further provided with interfacing means 15 for interfacing with a computer device. As computer device a laptop, desktop or also a palmtop computer can be used. The communication with the computer device can, for instance, be with infra red technology, known as such. In this case the interfacing means 15 are connected with an infra red transmitting and receiving member 16.

As shown in FIG. 3, the infra red transmitting and receiving means 16 of the data managing device 3 can communicate with infra red transmitting and receiving means 17 of the palmtop computer 20. The palmtop computer 20 will be provided with a program for storing and analyzing data received from the data managing device 3 and also for programming the data managing device 3.

The data managing device 3 furthermore can be provided with communication interfacing means 18. These communication interfacing means can for example cooperate with a palmtop 20 provided with a GPS telephone or such.

It is also possible to provide the data managing device 3 as shown in FIG. 4 with communication interfacing means 18 that contain GSM communication means. The data managing device 3 is provided with an antenna 9, so as to be capable of communicating directly with a remote station, capable of receiving data from the data managing device or sending instructions to be performed by the data managing device.

It is noted that the assembly according to the invention can be embodied in a great number of ways for a great number of different applications.

A more detailed description of the assembly 1, shown in FIG. 1, is given herein after.

The assembly 1 comprises at least a catheter 2 and a portable data managing device 3. The catheter is designed as a usual central venous catheter, but contains extra features, which enable on-line continuous determination of important characteristics of the heart and blood. To facilitate these extra features the catheter is connected with a portable electronic box, the data managing device 3. The data managing device 3 is an electronic diagnostic and storage device with a small display screen 6. Furthermore, the data managing device 3 can be compatible with any commercially available notebook, palmtop, cellular phone or alike.

As a usual central venous catheter, the catheter 2 has 2 or 3 lumens 8 for administration of medication, nutrients and/or different fluids as well as for central venous pressure measurement. Introduction of the catheter 2 is the same as for the usual central venous catheter by the antebrachial vein or by a jugular or subclavian vein approach. One of the features of the catheter is the presence of 2 or 4 electrodes at the distal end (see FIG. 2). These electrodes 7 enable continuous impedance measurement in the blood (see patent application: PCT/NL00/00378) by which precise determination can be obtained of the hematocrit in the blood and blood viscosity can be estimated with help of the data managing device 3. The same electrodes 7 enable continuous on-line registration of the intracavitary ECG signal, which also can be displayed and stored in the data managing device 3. Furthermore, at the distal part 5 of the catheter 2 a small sensor 9 for continuous on-line measurement of blood temperature is built in and temperature changes within 0.1 Celsius degree are displayed and stored in the data managing device 3.

All wires originating from the electrodes 7 at the tip 5 and the distal thermistor 9 are separately shielded, which enables application of electric current frequencies between 20 and 20.000 kHz without significant disturbance by stray capacitance. Despite these extra wires and the 2 or 3 lumens, the size of the catheter 2 can be not more than 7 French.

The distal electrodes 7, which register the intracavitary ECG, can also be used as markers of correct positioning of the catheter 2. As soon as the tip 5 of the catheter 2 enters the right atrium the appearance of the intracavitary ECG on the display screen 6 of the data managing device 3 will prevent mal positioning of the catheter 2 inside the right ventricle. The P-wave of the atrial depolarization on the intracavitary ECG should always be larger than the QRS-complex of the ventricular electrical activation. The characteristics of the P-wave are used as indicators for the positioning of the catheter 2. Interpretation of the P-wave prevents the entrance of the catheter 2 in the right ventricle and therefore enhances the possibility to avoid the potential dangerous ventricular arrhythmias that occur when a venous catheter enters the right ventricle.

If necessary, the introduction sheath of the catheter 2 remains positioned at the venous puncture site, where a flexible plastic isolation will cover part of the outside catheter 2. This plastic coverage enables repositioning of the catheter 2 under still sterile conditions, if necessary.

The data managing device 3 is specially designed as a portable device for connection between the catheter 2 and any commercially available notebook, palmtop 20, cellular phone or alike. Dimensions of the data managing device 3 enable positioning thereof in a small pocket in the pajama of a patient. The data managing device 3 is able to produce electrical currents at frequencies between 20 and 20.000 kHz as a sweep or as different sequential frequencies. Impedance values such as electrical resistivity and capacitance are displayed on the small screen 6 and can be stored. In order to measure impedance values in similar conditions of blood flow, the impedance can be measured by means of a repetitive mechanism of stimulate-and-sense signals with measuring intervals between 8 and 20 mS. The impedance measurements can be triggered on the intracavitary ECG signal, which is determined simultaneously. In case of a complete frequency sweep, the final result will be calculated during several cardiac cycles.

The data managing device 3 contains acoustic and optic alarm-systems, which alert the non-experienced user when heart rhythm, intracavitary ventricular ECG-signals, temperature or special blood characteristics surpass certain limits. These limits can be adapted and are displayed on the screen 6. Also an alarm will start as soon as the batteries are low. In order to minimize the dimensions and the battery power of the data managing device, preferably the data managing device is not designed for data storage of longer than 24-36 hours. The idea of the Data managing device is to play a role as a temporary portable electronic interface between heart and blood on one side and a hand-held computer on the other side. Within 24-36 hours the data in the data managing device should be downloaded on a hand held computer 20 and this downloading is so user-friendly, that all patients can manage. The hand held computer 20 will give full digital disclosure and analysis of the actual data of a patient and can compare these data with former information of the patient, if necessary with information of a year ago. In fact, the hand held computer 20 will contain the patient dossier in an electronic form. By a wired line or by WAP-technology physicians or other health care workers can download the information they want for their daily or weekly patient care. The downloaded information from the hand-held computer 20 may also digitally be sent to the doctor by phone or by internet (tele-medicine).

The thermistor 9 at the distal end of the catheter 2 will register fever or subfebril temperature. This will alert the patient to see the doctor, not only for possible new disease or exacerbation of an existing disease, but also to check whether the catheter itself has become infected and should be withdrawn. However, the thermistor 9 can also be used to guide cooling therapy, which is currently being used in intensive care units after successful resuscitations in order to reduce brain damage.

Preferably, the data managing device 3 has a special feature that can be connected with a portable injection system. These portable injection systems are already commercially available and are used in ambulatory patients in the hospital or at home for periodic drug and/or liquid administration. The data managing device 3 will be able to monitor the time-intervals at which periodically automatic treatment is given and an alarm will start as soon as too long or too short administration of liquids and/or drugs has been given.

Another favourable feature is the connection of the data managing device 3 with a non-invasive blood pressure measurement system; the pressure data can also be stored in the data managing device and be combined with the other parameters of the heart and blood, determined at the same time.

Permanent software upgrading of the data managing device 3 is possible and is meant to amplify its role as a temporary portable electronic interface between heart and blood and a hand-held computer. Having access to blood any time through the catheter 2, sophisticated analysis by chemical chips may be introduced in the data managing device 3 for determination of glucose, CRP, anticoagulation level, etc. Again the data can be analyzed immediately and the screen display 6 will show the result and the normal limits; if necessary, the patient may contact a health worker or the data will be stored and downloaded afterwards in the hand-held computer 20.

Having mentioned the several special and preferred features of the catheter and the data managing device according to the invention, one can imagine which applications this assembly may have. Because of the accurate monitoring possibilities of this assembly, chronic patients may be dismissed from hospital earlier and may need to come back to hospital only for special diagnostic procedures such as MRI or CT-scan, etc. Chronic patients who need special care may be treated by home nurses, who can read out the electronic patient dossier and discuss the results with the doctors in hospital by internet. Because of the electronics, dimensions of the data managing device 3 are tiny and will almost not interfere with the daily activities of the patient. For example the intracavitary ECG detection will avoid the use of several external electrodes.

In the Western World millions of people suffer from chronic heart failure and repetitive hospitalizations are often necessary; as people become older heart failure will even become more prevalent. Hospitalizations may be prevented if medical treatment and adequate monitoring at home will become available. Often intravenous medical therapy is needed in these patients and with the catheter and data managing device intravenous drug administration, as well as central venous filling pressure besides on-line monitoring of different cardiac and blood parameters will be possible. Home nursing is preferred by most patients and health care costs will be reduced by reduction of hospitalizations. The catheter and data managing device can be positioned in a Portocath-device, which is meant to remain intravenously for several months.

In the hospital itself, the combined use of the catheter and the data managing device may be useful especially, when patients are transported from intensive care units to radiology departments for diagnostic procedures. No extensive monitoring systems have to be taken with the patient.

A catheter of the assembly according to the invention, with less extra features may be used as just a peripheral venous line, while it still may be connected with the data managing device, for example for only monitoring of drug and/or fluid administration and for instant analysis of blood by micro-chip technique.

The unique concept of combining a catheter with an electronic interface may also be used for urinary tract catheters.

1. Assembly, comprising:
a portable data managing device; and
a catheter including a tubular body with a distal end portion to be introduced into the body of a person and a proximal end portion, said catheter comprising at its distal end portion at least one sensing member and connecting means, extending to the proximal end portion, for connecting the sensing member to the data managing device, and said data managing device comprising control and processing means for controlling the at least one sensing member and for processing signals generated thereby.

2. The assembly of claim 1, further comprising computer interfacing means for interfacing the control and processing means with a computer device.

3. The assembly of claim 1, further comprising communication interfacing means for interfacing the control and processing means with a telecommunication device.

4. The assembly of claim 3, wherein the communication means is incorporated in the data managing device.

5. The assembly of claim 1, wherein the at least one sensing member and the control and processing means are designed for measuring impedance in a body fluid.

6. The assembly of claim 1, wherein the at least one sensing member and the control and processing means are designed for measuring temperature.

7. The assembly of claim 1, wherein the at least one sensing member and the control and processing means are designed for measuring pressure in a body fluid.

8. The assembly of claim 1, wherein the at least one sensing member and the control and processing means are designed for measuring the concentration of at least one chemical component in a body fluid.

9. The assembly of claim 1, wherein the at least one sensing member and the control and processing means are designed for measuring electrical tension.

10. The assembly of claim 5, wherein the at least one sensing member and the control and processing means are designed for measuring in a blood volume in a body of a person.

11. The assembly of claim 1, wherein the data managing device comprises data storage means for storing at least one of measurement signals and processed data.

12. The assembly of claim 1, wherein the data managing device comprises an alarm.

13. The assembly of claim 12, wherein the data managing device comprises data storage means for storing at least one of measurement signals and processed data and wherein the data managing device is programmed such that the alarm is activated in a case wherein the at least one of measured and processed signal obtains a preset critical value.

14. The assembly of claim 12, further comprising a battery as a power supply for the data managing device, wherein said data managing device is programmed such that the alarm is activated in a case where the battery is depleted to a predefined extent.

15. The assembly of claim 1, wherein the data managing device comprises data storage means for storing at least one of measurement signals and processed data and wherein the data management device comprises a display and is programmed such that relevant information referring to the at least one of measured and processed signals is displayable by the display.

16. The assembly of claim 1, further comprising an injecting device having an injection fluid supply and at least one supply device connected with a lumen in the catheter, wherein the data managing device comprises data storage means for storing at least one of measurement signals and processed data and wherein the data managing device is programmed such that the at least one supply device is activated in response to the magnitude of the at least one of measured and processed signals.

17. The assembly of claim 6, wherein the at least one sensing member and the control and processing means are designed for measuring in a blood volume in a body of a person.

18. The assembly of claim 7, wherein the at least one sensing member and the control and processing means are designed for measuring in a blood volume in a body of a person.

19. The assembly of claim 8, wherein the at least one sensing member and the control and processing means are designed for measuring in a blood volume in a body of a person.

20. The assembly of claim 9, wherein the at least one sensing member and the control and processing means are designed for measuring in a blood volume in a body of a person.
